(19) United States
(12) Patent Application Publication
(10) Pub. No.: US 2021/0403871 A1
(43) Pub. Date: Dec. 30, 2021

(54) METHODS OF PRODUCING POPULATIONS OF MESENCHYMAL STEM CELLS FROM PERIPHERAL BLOOD AND USES THEREOF

(71) Applicant: INNO-HEALTH TECHNOLOGY CO., LTD., Taichung (TW)

(72) Inventor: Joun-Chaung HSIUNG, Kaohsiung City (TW)

(73) Assignee: INNO-HEALTH TECHNOLOGY CO., LTD., Taichung (TW)

(21) Appl. No.: 16/766,280

(22) PCT Filed: Nov. 28, 2017
(86) PCT No.: PCT/CN2017/113305

§ 371 Date: May 21, 2020

(51) Int. Cl. C12N 5/0775; A61K 35/28

(52) U.S. Cl. CPC C12N 5/0665; C12N 2500/35; A61K 35/28

(57) ABSTRACT

Disclosed herein are methods for providing a population of undifferentiated human mesenchymal stem cells (hMSCs). The methods include steps of, obtaining peripheral blood from a donor, adding glycerin to the peripheral blood, separating hMSCs from other somatic stem cells in the peripheral blood, and culturing the hMSCs in a medium containing glycerin thereby generating the population of undifferentiated hMSCs suitable for transplantation.

### BACKGROUND OF THE INVENTION

### 1. Field of the Invention

The present disclosure relates to methods of isolating and expanding mesenchymal stem cells (MSCs) and cell populations derived therefrom.

### 2. Description of Related Art

Mesenchymal stem cells (MSCs) may be isolated from various tissues including but not limiting to neonatal tissue (e.g., Wharton's jelly of human umbilical cord), bone marrow, peripheral blood, placenta, cord blood, and adipose tissue. Among them, peripheral blood is the easiest source for obtaining MSCs. However, MSCs are present at very low levels in peripheral blood and the art is limited by the inability of harvesting sufficient amount of MSCs for subsequent differentiation and uses, even if they are in virto expanded.

Therefore, there exist in the related art a need for an improved method for obtaining MSCs from peripheral blood, which allows the preparation of enriched MSCs populations sufficient for subsequent therapeutic uses.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The present invention in general relates to the discovery that mesenchymal stem cells (MSCs) may be isolated from peripheral blood of a human subject and expanded in vitro in a medium containing glycerin, thereby give rise to a substantially pure population of isolated undifferentiated human MSCs (hMSCs) suitable for autologous transplantation.

Accordingly, the first aspect of present disclosure is directed to a method of providing a population of undifferentiated hMSCs. The method comprises steps of:
(a) obtaining a peripheral blood from a human subject;
(b) adding glycerin to the peripheral blood in the step (a);
(c) separating hMSCs relative to other somatic stem cells in the peripheral blood of the step (b); and
(d) culturing the separated hMSCs of the step (c) in a medium containing glycerin,

thereby generating the population of undifferentiated hMSCs;

wherein the medium is devoid of any mobilization agent selected from the group consisting of, granulocyte colony stimulating factor (G-CSF), granulocyte-macrophage colony stimulating factor (GM-CSF) and a combination thereof.

According to embodiments of the present disclosure, in the step (a), the peripheral blood is collected in a tube devoid of any hemolytic agent.

According to embodiments of the present disclosure, in the step (b), the glycerin is present in the peripheral blood at a concentration of about 1-10 mg/mL.

According to embodiments of the present disclosure, in the step (d), the medium comprises at least 20% serum (v/v), and the glycerin is present in the medium at a concentration of about 0.1-1 mg/mL and the hMSCs is cultured in a culture plate containing the glycerin-containing medium at 37° C. for at least 12 hrs.

According to optionally embodiments of the present disclosure, the method further comprises,
(e) transferring the cultured hMSCs to another culture plate having a surface area at least 16 folds larger than that in the step (d) and continue to culture in the glycerin-containing medium for at least 4 days, to generate the population of undifferentiated hMSCs.

According to embodiments of the present disclosure, in the step (e), the hMSCs are cultured for at least 6 days to generate the population of undifferentiated hMSCs.

According to embodiments of the present disclosure, the population of undifferentiated hMSCs thus generated are negative for CD34−, CD45−, and HLA-DR cell surface markers, and are positive for CD73+, CD90+, and CD 105+ cell surface markers.

Accordingly, the present disclosure relates to the discovery of a clonal cell line comprising a substantially pure population of undifferentiated hMSCs prepared in accordance with the method of the present disclosure.

Further, the present disclosure also relates to the discovery of the use of the population of undifferentiated hMSCs or differentiated progeny thereof, which is prepared by the method of the present disclosure, for the treatment of a disease or a disorder of a subject in need thereof.

According to embodiments of the present disclosure, the population of hMSCs are substantially pure hMSCs.

According to embodiments of the present disclosure, the substantially pure hMSCs may be induced to differentiate into chrondrocytes, cartilage and adipocytes.

Accordingly, a method of treating a disease or a disorder of a subject in need of a transplantation of autologous human mesenchymal stem cells (hMSCs) is provided. The method comprises administering an effective amount of a population of undifferentiated hMSCs prepared in accordance with the method of present disclosure to the subject to treat the disease or disorder.

According to embodiments of the present disclosure, the disease or disorder treatable by the autologous transplantation of hMSCs is selected from the group consisting of a bone or cartilage disease, a neurodegenerative disease, a cardiac disease, a hepatic disease, a cancer, an autoimmune disease, graft versus host disease (GvHD), and wound healing and tissue regeneration.

According to certain embodiments of the present disclosure, an effective amount of the population of undifferentiated hMSCs or differentiated progeny thereof is administered to the subject for a duration of 3 days or less, for a duration of 2 days or less, or for the duration of 1 day or less.

Many of the attendant features and advantages of the present disclosure will becomes better understood with reference to the following detailed description considered in connection with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present description will be better understood from the following detailed description read in light of the accompanying drawings, where:

FIGS. 1A to 1C respectively illustrate the hMSCs of Example 1 that are positive for (A) CD90, (B) CD 105, and negative for (C) CD34 in accordance with one embodiment of the present disclosure;

FIG. 2A is a photograph depicting the chrondocytes differentiated from hMSCs of Example 1 confirmed by staining alkaline phosphatase in accordance with one embodiment of the present disclosure;

FIG. 2B is a photograph depicting the cartilage differentiated from hMSCs of Example 1 stained by Alcian Blue in accordance with one embodiment of the present disclosure; and

FIG. 2C is a photograph depicting the adipocytes differentiated from hMSCs of Example 1 stained by oil red O in accordance with one embodiment of the present disclosure.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

1. Definitions

For convenience, certain terms employed in the specification, examples and appended claims are collected here. Unless defined otherwise, all technical and scientific terms used herein have the same meaning as commonly understood by one of the ordinary skill in the art to which this invention belongs.

The singular forms “a”, “and”, and “the” are used herein to include plural referents unless the context clearly dictates otherwise.

The term “stem cells” is used in broad sense and includes traditional stem cells, progenitor cells, pre-progenitor cells and the like. The term “stem cell” refers to an undifferentiated cell capable of proliferation and giving rise to more progenitor cells having the ability to generate a large number of mother cells that in turn can give rise to differentiated, or differentiable daughter cells. The daughter cells themselves can be induced to proliferate and produce progeny that subsequently differentiate into one or more mature cell type. The term “stem cell” thus refers to a cell with the capacity or potential, under particular circumstances, to differentiate to a more specialized or differentiated phenotype, and which retains the capability, under certain circumstances, to proliferate without substantially differentiating.

The term “mesenchymal stem cells (MSCs)” refers to pluripotent stem cells capable of differentiating into more than one specific type of connective tissues, such as adipose, osseous, stroma, cartilaginous, elastic and fibrous connective tissues. For purpose of identification, human MSCs can be identified based on phenotype marker expression of CD34⁻, CD45⁻, CD73⁺, CD90⁺, and CD105⁺; and the ability to differentiate into tissues which support specially elements, including but not limiting to, chrondrocytes, cartilage and adipocytes.

The term “a population” with respect to an isolated population of hMSCs refers to a population of hMSCs that has been removed and separated from a mixed or heterogeneous population of cells. In some embodiment, the population of hMSCs is a substantially pure population of hMSCs, as compared to the heterogeneous population from which the cells are isolated or enriched from.

The term “substantially pure” with respect to a particular cell population, refers to a population of cells that is at least about 70%, preferably about 80%, more preferably about 90%, and most preferably about 95% pure, with respect to the cells making up a total cell population. The term “substantially pure” with regard to a population of hMSCs prepared in accordance with the method described in the present disclosure, refers to a population of hMSCs that contains fewer than about 30%, more preferably fewer than about 20%, 15%, 10%, 8%, most preferably fewer than about 5%, 4%, 3%, 2%, 1% or less than 1% of other non-hMSCs. According to some embodiments, the present disclosure encompasses methods to expand a population of hMSCs isolated from peripheral blood, in which the expanded population of peripheral blood derived hMSCs is a substantially pure population of hMSCs.

The term “clonal cell line” refers to a cell lineage that can be maintained in culture and has the potential to propagate indefinitely. A clonal cell line can be a stein cell line (e.g., hMSCs derived from peripheral blood of the present disclosure) or be derived from the peripheral blood derived hMSCs, and where the clonal cell line is used in the context of a clonal cell line comprising the hMSCs derived from peripheral blood of the present disclosure, which have been cultured under in vitro condition that allows proliferation without differentiation for at least 1 month. Such clonal stem cell lines (e.g., hMSCs derived from peripheral blood of the present disclosure) can have the potential to differentiate along several lineages of the cells from the original stem cell.

In the context of cell ontogeny, the adjective “differentiated” is a relative term. A “differentiated cell” is a cell that has progressed further down the developmental pathway than the cell it is being compared with. Thus, stem cells can differentiate to lineage-restricted precursor cells, which in turn can differentiate into other type of precursor cells further down the pathway, and then to the end-stage differentiated cell, that plays a characteristic roll in certain tissue type, and may or may not retain the capacity to proliferate further.

The term “treatment” as used herein are intended to mean obtaining a desired pharmacological and/or physiologic effect, e.g., tissue regeneration. The effect may be prophylactic in terms of completely or partially preventing a disease or symptom thereof and/or therapeutic in terms of a partial or complete cure for a disease and/or adverse effect attributable to the disease. “Treatment” as used herein includes preventative (e.g., prophylactic), curative or palliative treatment of a disease in a mammal, particularly human; and includes: (1) preventative (e.g., prophylactic), curative or palliative treatment of a disease or condition (e.g., a wound) from occurring in an individual who may be pre-disposed to the disease but has not yet been diagnosed as having it; (2) inhibiting a disease (e.g., by arresting its development); or (3) relieving a disease (e.g., reducing symptoms associated with the disease).

The term “administered,” “administering,” or “administration,” or “transplanting,” are used interchangeably herein to refer any appropriate route of delivery, including, without limitation, intraveneously, intramuscularly, intraperitoneally, intraarterially, intracranially, or subcutaneously administering a population of hMSCs of the present invention to a desired location in the subject, where at least a portion of hMSCs remain viable. The period of viability of the cells after the administration to a subject can be as sort as a few hours, e.g., 24 hours, to a few days, to as long as few months.

The term “an effective amount” as used herein refers to an amount of hMSCs effective, at dosages, and for periods of time necessary, to achieve the desired result with respect to the treatment of a disease resulted from platelet aggregation. For example, in the treatment of a wound, an agent (i.e., the hMSCs of the present disclosure) which facilitate the regeneration of skin and related soft tissue would be effective. An effective amount of an agent is not required to cure a disease or condition but will provide a treatment for a disease or condition such that the onset of the disease or condition is delayed, hindered or prevented, or the disease or condition symptoms are ameliorated. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the like. The effective amount may be divided into one, two or more doses in a suitable form to be administered at one, two or more times throughout a designated time period.

The term “subject” or “patient” is used interchangeably herein and is intended to mean a mammal including the human species that is treatable by the compound of the present invention. The term “mammal” refers to all members of the class Mammalia, including humans, primates, domestic and farm animals, such as rabbit, pig, sheep, and cattle; as well as zoo, sports or pet animals; and rodents, such as mouse and rat. Further, the term “subject” or “patient” intended to refer to both the male and female gender unless one gender is specifically indicated. Accordingly, the term “subject” or “patient” comprises any mammal which may benefit from the treatment method of the present disclosure. Examples of a “subject” or “patient” include, but are not limited to, a human, rat, mouse, guinea pig, monkey, pig, goat, cow, horse, dog, cat, bird and fowl. In a preferred embodiment, the subject is a human.

The term “medium” refers to a medium for maintaining a tissue or cell population, or culturing a cell population (e.g., culture medium), which contains nutrients that maintain cell viability and support proliferation. The cell culture medium contains any of the followings in an appropriate combination: salts, buffers, amino acids, glucose or other sugars, antibiotics, serum or serum replacement, and other components such as growth factors, etc. Cell culture medium ordinarily used for particular cell types are known to those skill in the art.

The term “mobilization” as used herein refers to the process whereby the cells leave their residing niches (e.g., bone marrow) and enter the blood. The term “mobilization agent” thus refers to an agent that results in the loss of adhesiveness of pools or populations of stem cells (e.g., MSCs) residing in stem cell niches in the peripheral tissue and the bone marrow.

2. The Isolation and Cultivation of Human Mesenchymal Stem Cells (hMSCs) Derived from Peripheral Blood

Mesenchymal stem cells (MSCs) are non-hematopoietic stem cells that are normally obtained from bone marrow. MSCs are present at very low levels in peripheral blood and harvesting from peripheral blood often does not produce sufficient amount of cells, even if they are in virto expanded, for therapeutic uses. The inventor of the present study unexpectedly discover methods for isolating and expanding MSCs derived from peripheral blood, thereby give rise to a substantially pure population of hMSCs that can be used either alone or in combination with other cells for future autologous therapeutic applications in regenerative therapy, such as wound healing and bone repair, and other orthopedic indications.

Accordingly, the first aspect of the present disclosure aims to provide a method of providing a population of undifferentiated hMSCs. The method comprises:
(a) obtaining a peripheral blood from a human subject;
(b) adding glycerin to the peripheral blood in the step (a);
(c) separating hMSCs relative to other somatic stem cells in the peripheral blood of the step (b); and
(d) culturing the separated hMSCs of the step (c) in a medium containing glycerin, thereby generating the population of hMSCs;
   wherein the medium is devoid of any agent selected from the group consisting of granulocyte colony stimulating factor (G-CSF), granulocyte-macrophage colony stimulating factor (GM-CSF), and a combination thereof.

According to some embodiments, in the step (a) of the present method, peripheral blood is drawn from a human subject and collected in a tube containing an anti-coagulant (e.g., heparin). According to embodiments of the present disclosure, the subject may be a healthy subject or a subject suffering from a disease or a disorder that requires transplantation of hMSCs. Further, there is no particular age limitation on the subject from which peripheral blood is drawn for the preparation of present hMSCs. According to certain embodiments, peripheral bloods are drawn from human subjects respectively age between 20 to 30. According to other embodiments, peripheral bloods are drawn from human subjects respectively age between 50 to 60. According to further embodiments, peripheral bloods are drawn from human subjects respectively age between 70 to 80.

Immediately after peripheral blood is collected, it is mixed with glycerin, and incubates at 37° C. for at least 4 hrs, preferably at least 6 hrs (the step (b) of the present method). According to embodiments of the present disclosure, glycerin is preferably present in the peripheral blood in the amount of about 1-10 mg/mL, such as 1, 2, 3, 4, 5, 6, 7, 8, 9, and 10 mg/mL; more preferably about 2-8 mg/mL, such as 2, 3, 4, 5, 6, 7, and 8 mg/mL; most preferably about 5 mg/mL.

After incubation, hMSCs in the glycerin containing peripheral blood are separated from other somatic stem cells by suitable means, such as a cell sorter, or centrifugal elutriation (the step (c) of the present method).

According to some embodiments of the present disclosure, in the step (c) of the present method, hMSCs in the peripheral blood are separated from other somatic stem cells by a cell sorter, such as a laser scanning Flow Cytometer. A Flow Cytometer typically consists of a laser light source, flow measurement chamber, and an optical system consisting of lenses, filters, and light detectors. Two photo-multiplier tubes, one at 180 degrees and one at 90 degrees to the laser, are used to measure forward (FSC) and right-angle scatter (SCC), respectively. Three fluorescence detectors, each consisting of a filter and photomultiplier in the forward direction tube, are used to detect fluorescence. Fluorescence activated cell sorting (FACS) machine is set in a way that cells of a particular forward scatter and/or side scatter are selected. FSC is proportional to cell surface area or size. FSC is a measurement of mostly diffracted light and is detected just off the axis of the incident laser beam in the forward direction by a photodiode. FSC provides a suitable method of detecting particles greater than a given size independent of their fluorescence. Side-scattered light (SCC) is proportional to granularity or internal complexity. SSC is a measurement of mostly refracted and reflected light that occurs at any interface within the cell where there is a change in refractive index. SSC is collected at approximately 90 degrees to the laser beam by a collection lens and then redirected by a beam splitter to the appropriate detector. Thus, cells may be selected by gating at particular FSC and SSC. According to working embodiments of the present disclosure, hMSCs in the peripheral blood are separated from other somatic stem cells based on the level of cell surface expression, in which cell populations gated for events stained with different fluorescence intensities are selected.

Optionally or in addition, in the step (c), the glycerin containing peripheral blood obtained in the step (b) is drawn into a cell sorter such as COBE Spectra Apherasis System, and optionally, an anti-coagulant is added to keep the blood from clotting during the procedure. The mixture is then cycled through a centrifuge to separate the peripheral blood into a MSC population and mononuclear cells from other blood components and plasma. The thus separated MSCs are then pumped into a collection bag for storage, while other blood components and plasma are discarded or returned to the subject.

Optionally or in addition, in the step (c) of the present method, hMSCs in the glycerin containing peripheral blood are separated from other somatic stem cells by centrifugal elutriation where smaller stem cells are fractioned from stem cells that are larger in size. In certain embodiments, the glycerin containing peripheral blood in the step (b) is introduced into a generally funnel-shaped separation chamber located in a spinning centrifuge. A flow of liquid elutriation buffer is introduced into the chamber containing the peripheral blood. As the flow rate of the liquid elutriation buffer solution in increased through the chamber, the liquid sweeps smaller sized, slower-sedimenting cells toward an elutriation boundary within the chamber, while larger, fast-sedimenting cells migrate to an area of the chamber where the centrifugal force and the sedimentation forces are balanced. Accordingly, as the peripheral blood comprises many different populations of stem cells, they may be separated into distinct populations using elutriation, where smaller stem cells are fractioned from stem cells that are larger in size. Any elutriation device may be used to obtain and/or isolate MSCs derived from peripheral blood.

In the step (d) of the present method, the hMSCs thus produced in the step (c) are further expanded by subjecting them to cultivation in condition that enhances proliferation of the isolated hMSCs. According to preferred embodiments of the present disclosure, the hMSCs thus obtained in the step (c) are cultivated in petri dishes (surface area about 10 cm²) in a growth medium comprising glycerin, and devoid of any known stem cell mobilization agent, including but not limiting to, granulocyte colony stimulating factor (G-CSF), granulocyte-macrophage colony stimulating factor (GM-CSF), and a combination thereof. It will be appreciated that when referring to a medium devoid of a particular component, the present disclosure contemplates that the medium comprises this component, but at a concentration that is below its minimal activity. Thus, for example, certain medium may comprise trace amounts of stem cell mobilization agent described above (i.e., G-CSF, GM-CSF or a combination thereof), however, the methods of the present disclosure relate to a medium being devoid of exogeneously added growth factor beyond which is included in a commercial medium's formula, or that resulting from overall adjustment of medium component concentration.

A typical medium suitable for cultivating hMSCs of the present invention may be a complete medium (e.g., DMEM) containing at least 20% (v/v) serum, preferably at least 30% (v/v) serum, more preferably at least 50% serum, in which glycerin is present at a concentration preferably about 0.1-1.0 mg/mL, such as 0.1, 0.2, 0.3, 0.4, 0.5, 0.6, 0.7, 0.8, 0.9 and 1.0 mg/mL; more preferably about 0.2-0.8 mg/mL, such as 0.2, 0.3, 0.4, 0.5, 0.6, 0.7, and 0.8, mg/mL; most preferably about 0.5 mg/mL. In some embodiments, 100% (v/v) serum, in which 0.1-1.0 mg/mL glycerin may be added, is used as a growth medium for cultivating hMSCs. During the culture, supplements required for cell metabolisms may be added to the medium, such as amino acids, vitamins, minerals, and useful proteins such as transferrin, and the like. The medium may also contain antibiotics to prevent contamination with yeast, bacteria, and fungi, such as penicillin, streptomycin, gentamicin, and the like. According to one embodiment of the present disclosure, the culture medium comprises pituitary gland extract, plasma and fetal bovine serum. The culturing may be effected for a period of time to allow the expansion of hMSCs, so that enough quantity of hMSCs are produced. According to one specific embodiment, the hMSCs obtained in the step (c) are cultivated in a medium containing 30% (v/v) serum, and 0.5 mg/mL of glycerin, at 37° C. for at least 12 hrs, preferably at least 16 hrs, more preferably at least 24 hrs, thereby give rise to the population of hMSCs suitable for autologous transplantation.

According to embodiments of the present disclosure, the population of hMSCs produced in the present method exhibit negative staining for the hematopoietic stem cell markers CD34, and CD45, as well as the human leukocyte antigen—Antigen D related (HLA-DR) cell surface marker that known to mediate graft-versus-host disease (GvHD); and positive staining for CD73, CD90, and CD 105 cell surface markers. Methods of determining cell surface marker expression are well known in the art. Examples include immunological methods such as FACS, and biochemical methods (e.g., cell surface labeling via radioactive, fluorescence, or avidin-biotin). Alternatively, magnetic cell sorting (MACS) or immunopanning may be used to identify the cells.

The expression of the pattern of cell surface markers on the population of hMSCs produced by the present method confirms that they are indeed enriched population of undifferentiated mature hMSCs, which may be used in regenerative therapy that requires autologous stem cells transplantation.

3. Human MSC Clonal Cell Line

Another aspect of the present disclosure thus is directed to a clonal cell line comprising a substantially pure population of undifferentiated hMSCs prepared in accordance with any of the method described above.

According to this aspect of the present disclosure, the hMSCs freshly isolated or being produced by the method described above are further expanded by cultivating under conditions that do not induce differentiation, such as in the absence of any differentiation factors. In general, after hMSCs are isolated from their host or being selected to produce a population of hMSCs (e.g., the hMSCs thus produced in the step (d) of the present method), which may be a heterogeneous population of cells or a purified population of hMSCs, they are subject to further cultivation in a culture plate having a surface area at least 16 folds, at least 17 folds, at least 18 folds, or at least 20 folds larger than that in the step (d), and continue culturing in the glycerin-containing medium described above (i.e., the medium in the step(d)). Optionally or alternatively, the hMSCs are cultivated in a bioreactor so as to produce a large number of cells. According to embodiments of the present disclosure, the medium suitable for use in the present step is devoid of any cell mobilization agent such as G-CSF, GM-CSF and a combination thereof; and comprises glycerin at a concentration preferably about 0.1-1.0 mg/mL, such as 0.1, 0.2, 0.3, 0.4, 0.5, 0.6, 0.7, 0.8, 0.9 and 1.0 mg/mL; more preferably about 0.2-0.8 mg/mL, such as 0.2, 0.3, 0.4, 0.5, 0.6, 0.7, and 0.8, mg/mL; most preferably about 0.5 mg/mL. The culture is preferably effected for a period of, at least 7 days, preferably at least 10 days, and more preferably at least 14 days, to generate a homogeneous population of undifferentiated hMSCs, which comprise preferably, at least 70% hMSCs, more preferably at least 80% hMSCs, and most preferably at least 90% of hMSCs.

According to embodiments of the present disclosure, the hMSCs are allowed to expand for at least 2 population doublings, at least 4 populations doublings, at least 6 populations doublings, at least 8 populations doublings, at least 10 populations doublings, at least 12 populations doublings, at least 15 populations doublings, at least 20 populations doublings, at least 30 populations doublings, or at least 40 populations doublings.

Similar to the finding in the step (d) above, the homogeneous population of hMSCs thus produced remains undifferentiated and exhibit negative staining for the hematopoietic stem cell markers CD34, and CD45, as well as HLA-DR cell surface marker; and positive staining for CD73, CD90, and CD 105 cell surface markers.

The cultured populations of hMSCs generated using the method described herein may be used freshly or stored until further use, such as cryopreserved in the presence of a cryopreservant, which includes but are not limited to, glycerol, dimethyl sulfoxide (DMSO), and the like.

4. Compositions Comprising the Present hMSCs

The hMSCs of the present disclosure may be provided per se, along with the culture medium, and combined with pharmaceutically acceptable carrier and other additional agent, which may promote cell engraftment and/or organ function (e.g., immunosuppression agents, antibiotics, growth factor, and the like). Hence, the hMSCs of the present disclosure may be administered to the subject in a pharmaceutical composition, where they are mixed with a pharmaceutically carrier or diluent, such as sterile saline and aqueous buffer solution.

Suitable routes for administration may include, but are not limited to, oral, rectal, transmucosal (e.g., transnasal), intestinal or parenteral delivery including intramuscular, subcutaneous, and intraventrically injection, as well as intrathecal, intraventricular, intraveneous, intraperitoneal, or intraocular injections.

Alternatively, one may administer the pharmaceutical composition in a local rather than systemic manner. For example, by injection of the pharmaceutical composition directly to the target site (e.g., an organ).

Pharmaceutical compositions for use in accordance with the present disclosure may be formulated in conventional manner using one or more pharmaceutically acceptable carriers. Proper formulation depends on the route of administration.

For injection, the hMSCs of the present disclosure may be formulated in aqueous solutions, preferably in physiologically acceptable buffers such as Ringer's solution or physiologically acceptable salt solution.

Pharmaceutical compositions suitable for use in the context of the present disclosure include compositions wherein the active ingredients (e.g., hMSCs) are contained in an amount effective to achieve intended purpose. Determination of the effective amount is well within the capability of those skill in the art, especially in light of the description of the present disclosure.

Pharmaceutical compositions of the present disclosure may be presented as a kit, which may contain one or more unit dosage forms containing the present hMSCs. The kit may further comprise a label or package insert on or associated with the kit. The label or package insert indicates that the kit is for treating certain diseases and/or disorders. Alternatively or additionally, the kit may further comprise a buffer, such as a phosphate-buffered saline, or a Ringer's solution. The kit may further include directions for how to administer the present hMSCs to an intended target site.

According to one embodiment, the kit may include, at least, (a) a first container containing the present hMSCs; and optionally, b) a second container containing a buffer, and (c) a legend associated with the kit for instructing a user how to use the kit. The legend may be in a form of pamphlet, tape, CD, VCD or DVD.

5. Uses of the Present hMSCs or Differentiated Progeny there of

The population of undifferentiated hMSCs generated by the present method described above or differentiated progeny thereof may be used for autologous transplantation to treat and/or prevent a myriad of diseases and/or disorders in which beneficially effects are achieved by autologous transplantation.

Thus, another aspect of the present disclosure is directed to a method of treating a disease and/or disorder in a subject, in which an effective amount of the population of hMSCs generated by the present method or differentiated progeny thereof, is administered to the subject for a duration ranging from several days to several weeks.

MSCs are capable of differentiating into various cell lineages. Differentiation may be achieved by culturing MSCs in a growth environment enriched for cells with desired phenotype, e.g., osteoblasts, adipocytes and etc. The culture may comprise agents that enhance differentiation to s specific lineage. According to one embodiment of the present disclosure, the population of hMSCs is differentiated into chrondrocytes, in which cells are cultured in a medium containing dexamethasone, ascorbic acid, insulin, transferrin and selenous acid, until confluence (see Williams et al. (2003) Tissue Engineering. 9(4), 679). The differentiated chrondrocytes are identified by staining alkaline phosphastase. According to another embodiment of the present disclosure, the population of hMSCs is differentiated into cartilage by subjecting them to culture in a commercial cartilagenic medium. The differentiated cartilages are confirmed by staining proteoglycans with Alcian Blue. According to a further embodiment of the present disclosure, the population of hMSCs is differentiated into or adipocytes. To induce adipogenic differentiation, hMSCs are treated with adipogenic medium, which contains hydrocortisone and indomethacin; alternatively or additionally, any commercial MSC adipogenic stimulatory supplement, such as those available from StemCell Technologies Inc (Vancouver, Calif.), may be used. Adipogenic differentiation is confirmed by oil-red staining.

According to embodiments of the present disclosure, the population of hMSCs or differentiated progeny thereof may be used to treat diseases and/or disorders selected from the group consisting of, a bone or cartilage disease, a neurodegenerative disease, a cardiac disease, a hepatic disease, a cancer, an autoimmune disease, graft versus host disease (GvHD), and wound healing and tissue regeneration.

Bone defects suitable for treatment using the population of hMSCs generated by the present method or differentiated progeny thereof include, but are not limited to, osteogenesis imperfecta, fracture, congenital bone defects, and the like.

The population of hMSCs generated by the present method or differentiated progeny thereof may also be implanted in a subject to provide osseous and connective tissue support for orthopedic and other (e.g., dental) prosthetic devices, such as joint replacements and/or tooth implants.

Since MSCs are capable of differentiating into cartilage, thus, the population of hMSCs generated by the present method or differentiated progeny thereof are suitable for treating joint conditions, which include but are not limited to, osteoarthritis, rheumatoid arthritis, inflammatory arthritis, chondromalacia, avascular necrosis, traumatic arthritis and the like.

The population of hMSCs generated by the present method may also be used to treat CNS disorders. Examples of CNS disorders include, but are not limited to, a pain disorder, a motion disorder, a dissociative disorder, a mood disorder, an affective disorder, a neurodegenerative disease, and a convulsive disorder. More specific examples of such disorders include, but are not limited to, Parkinson's disease, amyotrophic lateral sclerosis (ALS), multiple sclerosis, Huntington's disease, autoimmune encephalomyelitis, diabetic neuropathy, glaucomatous neuropathy, macular degeneration, action tremors and tardive dyskinesia, panic, anxiety depression, alcoholism, insomnia, manic behavior, Alzheimer's disease, and epilepsy.

MSCs are known to interact with hematopoietic stem cells and immune cells, and represent potential cellular therapy to enhance allogenic hematopoietic engraftment and prevent graft versus host disease (GvHD). Thus, the population of hMSCs generated by the present method may also be used to treat GvHD.

The population of hMSCs generated by the present method or differentiated progeny thereof may also be used to promote tissue regeneration. Transplantation of hMSCs is thus useful for treating autoimmune diseases, inflammatory diseases, acute and chronic ischemic conditions, tissue engineering, regenerating new tissues and naturally healing diseased or injured organs.

It is known that when MSCs are introduced into infarcted heart, they can prevent deleterious remodeling and improve recovery. MSCs have been injected directly into the infarct, or they have been administered intravenously and seen to home to the site of injury. Thus, the present population of hMSCs or or differentiated progeny thereof may also be used to treat a cardiac disease, particularly, cardiac infarction.

Examples of cancer that is treatable by the present population of hMSCs or differentiated progeny thereof include, but are not limited to, breast cancer, brain tumor, melanoma, lung cancer, lymphoma, neuroepithelioma, kidney cancer, prostate cancer, stomach cancer, colon cancer, rectal cancer, pancreatic cancer and uterus cancer. In someembodiments, the cancer is metastatic.

The population of hMSCs or differentiated progeny thereof can be administered to the treated individual using a variety of transplantation approaches, the nature of which depends on the site of implantation. The cells may be transplanted to a damaged or healthy region of the tissue. In the case when hMSCs are administered to a healthy region, they will then migrate to the damage region.

The population of hMSCs or differentiated progeny thereof can be transplanted by means of direct injection into an organ, injection into the bloodstream, intraperitoneal injection, injection directly into lymphoid organs. Suitable methods of transplantation can be determined by monitoring the homing and engraftment of the implanted cells to the desired organ, the expression of desired organ-specific markers, and the function of the derived organ of the subject.

The population of hMSCs or differentiated progeny thereof is administered in an amount that will achieve desired therapeutically effects in the subject. Effective dosage of hMSCs to be used in the present method can be estimated initially from in vivo and/or in vitro cell culture assay. For example, a dose can be formulated in experimental animals to achieve desirable concentration, such information is then used to more accurately determine human dosage. The dosage may vary depending on the route of administration. The exact dosage, and route of administration can be determined by the attending physician in view of the patient's condition and history.

Depending on the condition to be treated, the effective amount of the present hMSCs can be of a single dose or multiple dosages, with course of treatment lasting for a duration of several days to several weeks, or until cure is effective or symptoms of diseases are diminished.

Additional objectives, advantages and features of the present disclosure will become apparent to one ordinarily skilled in the art upon examination of the following examples, which are not intended to be limiting.

### EXAMPLES

Materials and Methods

Surface Antigen Analysis

Cells were detached from the culture plate by use of 0.25% trypsin-EDTA. The cells were washed with PBS solution containing 1% BSA, and stained at 4° C. for 30 min with fluorescein isothiocyanide (FITC) or phycoerythrin (PE) conjugated antibody. MSC markers: anti-CD29 (integrin (31 chain), anti-CD 105 (SH2, endoglinCD73 (+), and hematopoietic stem cell marker: anti-CD34 (negative control); and leukocyte marker: anti-CD45 (leukocyte common antigen). The cells were then washed in PBS and analyzed using a FACS calibur flow cytometer (FACSCanto, BD Biosciences, Becton, Dickinson and Company, San Jose, Calif.). The cells were passed at a rate of up to 1,000 cells/second, using a 488 nm argon laser beam as the light source for excitation. Cells stained with FITC and PE-conjugated isotype controlled antibodies were used to calculate the background fluorescence.

Induction of differentiation of hMSCs to bone cells using a STEMPRO osteogenesis differentiation kit (Gibco), and to adipocytes and chondrocytes using STEMPRO adipogenic differentiation kit (Gibco) and Chondrogenic induction medium (Gibco). Those cells were then maintained at 37 ° C. in a 95% air and 5% CO2 humidified incubator. Medium was changed every 2-5 days for the duration of 14 days. Differentiated cartilage was assess by Alcian Blue staining in accordance with standard procedures. Differentiated adipocytes were assessed by staining with oil-red-O in accordance with standard procedures.

### Example 1

### Preparation and Culture of Human Mesenchymal Stem Cells (hMSCs)

Fifteen human subjects were included in the present study with informed written consent. They were assigned into respective groups in accordance with their age: age 50-60, 70-80 or 20-30, with 5 subjects in each group. Blood (10 mL) was drawn from each subject, and collected in a tube containing heparin, 0.5 mL of glycerin (100 mg/mL) was then added, and the glycerin-containing blood was stored in an incubator at 37° C. for 6 hrs. Then, the glycerin-containing blood was centrifuged at a speed of 3,500 rpm for 18 min. After centrifugation, the cell in the middle layer of the tube was extracted and re-suspended in PBS solution (8 mL). The cell suspension was centrifuged again at the speed of 18×g for 13 min, discarded the supernatant, and the cells in the middle layer of the tube was extracted and seeded in a culture plate (surface area about 10 cm²) containing 10 mL thermo-life medium, which composed of: keratinocyte-SFM 500 mL, bovine pituitary extract 2.5 mL, 13% of human fresh frozen plasma (FFP), 30% fetal bovine serum, 15 mL of glycerin (100 mg/mL), and epidermal growth factor (EGF, 2.5 μg). The cell-seeded culture plate was then placed in an incubator at the condition of 37° C., 70% RH (relative humidity), 5% CO₂, for 16-18 hrs. The cells were then harvested and re-seeded in another culture plate (surface area about 175 cm²) containing 10 mL thermo-life medium as described above. The medium was replaced every 3 days, and the change of medium was repeated at least once. Mesenchymal stem cells started to appear on day 5, and eventually reached confluence on about day 7. The thus obtained hMSCs were then isolated and stored at frozen temperature until further use.

Further, it was found that hMSCs may be successfully obtained and expanded in vitro from peripheral blood of an aged subject, such as the subject that is between 70 to 80 years old.

### Example 2

### Characterization of the hMSCs of Example 1

2.1 Cell Surface Antigen Analysis

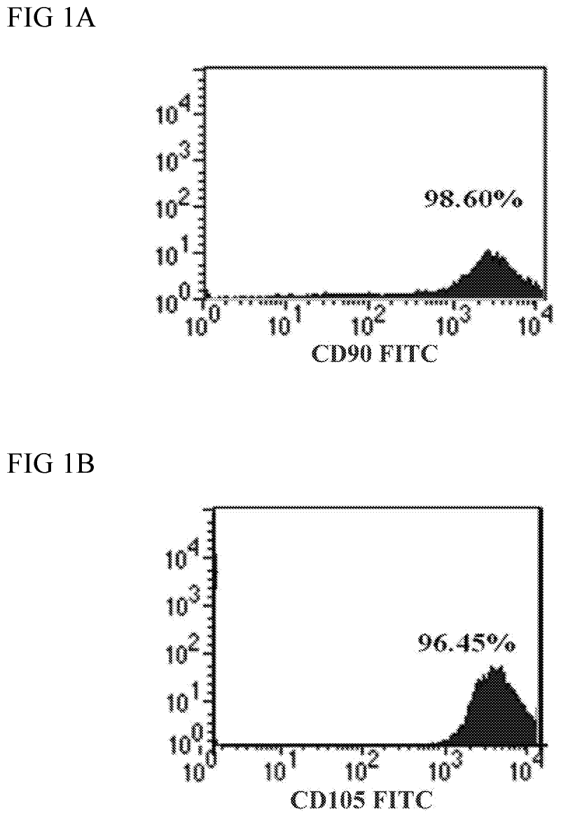

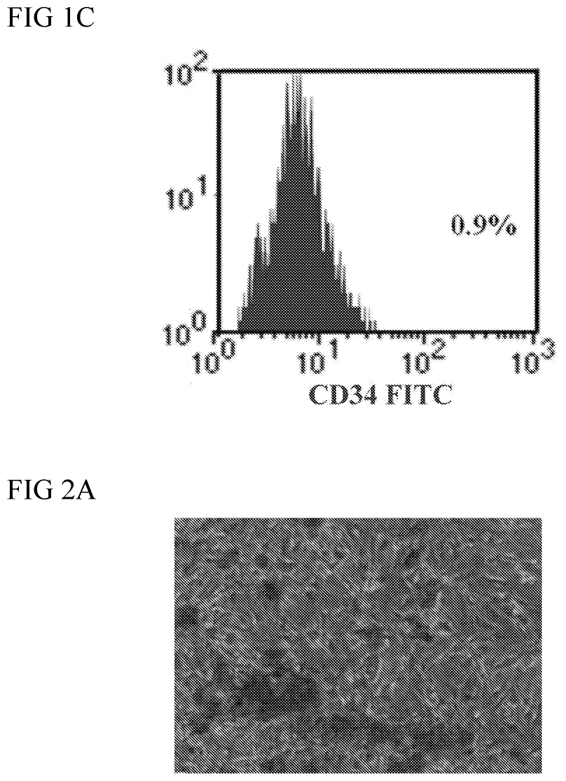

The adherent cells of Example 1 were isolated using trypsin/EDTA and subjected to surface antigen analysis in a flow cytometer in accordance with procedures described in “Materials and Methods.” Results are depicted in FIG. 1.

The cells of Example 1 were positive for CD73 (data not shown), CD90 (FIG. 1A), and CD 105 (FIG. 1B) cell surface markers, and negative for CD34 (FIG. 1C), CD45 (data not shown), and HLA-DR (data not shown); which confirmed they were indeed mesenchymal stem cells.

2.2 Differentiation of hMSCs of Example 1

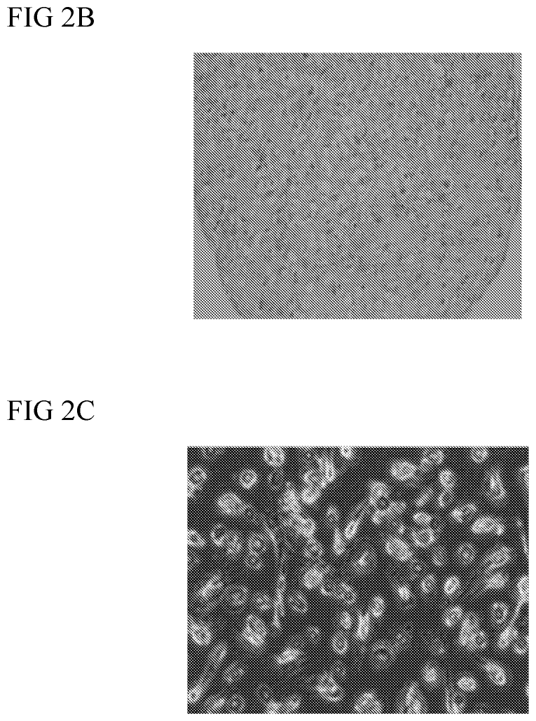

In this example, to substantiate that the hMSCs of Example 1 still possess the ability of differentiation, they were cultured in media respectively containing agents that trigger subsequent differentiation of hMSCs. The chrondrocytes, cartilage and adipocytes differentiated from hMSCs of Example 1 were confirmed by staining with alkaline phosphatase (FIG. 2A), Alcian Blue (FIG. 2B), and oil red O (FIG. 2C), respectively, according to standard procedures.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A method of providing a population of undifferentiated human mesenchymal stem cells (hMSCs) comprising:
(a) obtaining a peripheral blood from a human subject;
(b) adding glycerin to the peripheral blood in the step (a);
(c) separating hMSCs relative to other somatic stem cells in the peripheral blood of the step (b); and
(d) culturing the separated hMSCs of the step (c) in a medium containing glycerin, thereby generating the population of hMSCs;
wherein the medium is devoid of any mobilization agent selected from the group consisting of, granulocyte colony stimulating factor (G-CSF), granulocyte-macrophage colony stimulating factor (GM-CSF) and a combination thereof.

2. The method of claim 1, wherein in the step (a), the peripheral blood is collected in a tube devoid of any hemolytic agent.

3. The method of claim 1, wherein in the step (b), the glycerin is present in the peripheral blood at a concentration of about 1-10 mg/mL.

4. The method of claim 1, wherein in the step (d), the medium comprises at least 20% (v/v) serum, and glycerin is present in the medium at a concentration of about 0.1-1.0 mg/mL, and the hMSCs is cultured in a culture plate containing the glycerin-containing medium at 37° C. for at least 16 hrs.

5. The method of claim 4, further comprising:
(e) transfering the cultured hMSCs to another culture plate having a surface area at least 20 folds larger than that in the step (d), and continue to culture in the glycerin-containing medium to generate the population of undifferentiated hMSCs.

6. The method of claim 5, wherein in the step (e), the hMSCs are cultured for at least 4 days to generate the population of undifferentiated hMSCs.

7. The method of claim 4, wherein the population of undifferentiated hMSCs thus generated are negative for CD34−, CD45−, and HLA-DR cell surface markers, and are positive for CD73+, CD90+, and CD 105+ cell surface markers.

8. (canceled)

9. (canceled)

10. (canceled)

11. (canceled)

12. A method of treating a disease or a disorder of a subject comprising administering an effective amount of a population of undifferentiated hMSCs prepared in accordance with the method of claim 4 to the subject to treat the disease or disorder.

13. The method of claim 12, wherein the population of undifferentiated hMSCs prepared in accordance with the method of claim 4 is substantially pure population of hMSCs.

14. The method of claim 13, wherein the population of undifferentiated hMSCs can differentiate into chrondrocytes, cartilage or adipocytes.

15. The method of claim 12, wherein the disease or disorder is selected from the group consisting of a bone or cartilage disease, a neurodegenerative disease, a cardiac disease, a hepatic disease, a cancer, an autoimmune disease, graft versus host disease (GvHD), and wound healing and tissue regeneration.
